(19) United States
(12) Patent Application Publication
(10) Pub. No.: US 2004/0072903 A1
(43) Pub. Date: Apr. 15, 2004

(54) Carboxyalkylether-acat inhibitors combinations

(76) Inventors: Bruce Jeffrey Auerbach, Ann Arbor, MI (US); Donna Lee Zobel, Ann Arbor, MI (US)

Correspondence Address: Charles W Ashbrook, Warner- Lambert Company, 2800 Plymouth Road, Ann Arbor, MI, 48105, US

(21) Appl. No.: 10/182,747

(22) PCT Filed: May 8, 2001
(86) PCT No.: PCT/US01/14804

(51) Int. Cl. A61K 31/195

(52) U.S. Cl. 514/561; 514/599

(57) ABSTRACT

The invention is a pharmaceutical composition comprising a carboxyalkylether which lowers triglycerides and LDL and elevates HDL, and an ACAT inhibitor which improves dyslipidemias in mammals, said composition being useful for treating dyslipidenia and ischemic syndromes, and for preventing or delaying the onset of heart attacks. The carboxyalkylethers have the formula (1) wherein Y, and Y₂ include COOH, RI, R₂, R₃, and P, can be alkyl, and n and m are integers from 2 to 9; and the ACAT inhibitors have the formula (IV) where R is hydrogen, X is 0, RI and R₂ are substituted phenyl, and Y is alkylene.

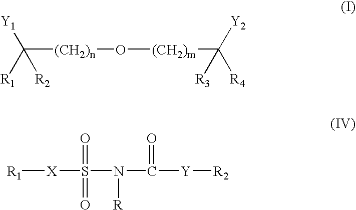

### FIELD OF THE INVENTION

This invention relates to combinations of carboxyalkylether compounds with compounds which inhibit acyl-coenzyme A:cholesterol acyltransferase, and to a method for treating dyslipidemias employing such combinations.

### BACKGROUND OF THE INVENTION

Heart disease remains one of the leading causes of death throughout the world. Recent evidence has established that reducing the bad form of cholesterol, namely low-density lipoprotein, or LDL, is important in improving ischemic syndromes and avoiding second and subsequent, and possibly even a first, heart attack. Several agents known as statins are now used clinically to lower LDL, and to treat and prevent ischemic syndromes.

There is evidence from animal models that compounds which inhibit the enzyme acyl-coenzyme A:cholesterol acyltransferase (ACAT) will be effective anti-atherosclerotic agents (Curr. Med. Chem., 1994;1:204-225). It is well- established that when the majority of cholesterol in plasma is carried on apolipoprotein B-containing lipoproteins, such as low-density lipoproteins (LDL-C) and very-low-density lipoproteins (VLDL-C), the risk of coronary artery disease in man is increased (Circulation, 1990;81 :1721-1733). Conversely, high levels of cholesterol carried in high-density lipoproteins (HDL-C) is protective against coronary artery disease (Am. J Med., 1977;62:707-714). Thus, a drug which reduces the levels of atherogenic LDL-C and VLDL-C and elevates levels of protective HDL-C will produce a less atherogenic lipoprotein profile and thus a beneficial effect on atherosclerotic disease and its complications. This beneficial effect was demonstrated in man in the Helsinki Heart Study with the lipid regulator gemfibrozil which decreased LDL-C, increased HDL-C, and reduced the incidence of coronary artery disease (N. Eng. J Med., 1987;317:1237-1245 and N. Eng. J Med, 1999;341:410-418).

Compounds which inhibit acyl-coenzyme A:cholesterol acyltransferase are known as ACAT inhibitors. Certain ACAT inhibitors and the methods for preparing them are taught in U.S. Patent Nos. 5,491,172 and 5,633,287, which are incorporated herein by reference. One such ACAT inhibitor currently undergoing clinical evaluation is avasimibe, also known as CI-1011 and PD-148515.

U.S. Pat. No. 5,648,387 discloses a group of compounds known as carboxyalkylethers. The compounds are reported to be useful for treating dyslipidemias and atherosclerosis. The compounds alter various forms of lipoprotein cholesterol; for example, they elevate HDL-C and lower LDL-C, and are thus useful for treating patients with, or at risk of developing, ischemic syndromes. They are also reported to increase insulin sensitivity. One carboxyalkylether currently undergoing clinical evaluation is CI-1027.

It has now been discovered that combination therapy with a carboxyalkylether and an ACAT inhibitor results in dramatic improvement in lipid control. Accordingly, such combinations are especially useful in treating dyslipidemias and associated complications such as ischemic syndromes, including heart attacks and related cardiovascular disorders.

### SUMMARY OF THE INVENTION

This invention provides a method of treating dyslipidemias and ischemic syndromes by administering to a subject in need of treatment a combination of a carboxyalkylether and an ACAT inhibitor.

Carboxyalkylethers are compounds characterized by having two alkyl groups linked through an oxygen atom, wherein the alkyl groups are substituted by a carboxy group or equivalent carboxy miimics. Typical carboxyalkylethers are those described in U.S. Pat. No. 5,648,387 (this and all other references cited are incorporated herein by reference); compounds of Formula 1

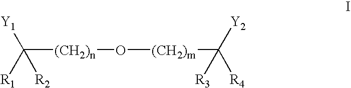

and the pharmaceutically acceptable salts thereof, wherein:

n and m independently are integers from 2 to 9;

R₁, R₂, R₃, and R4 independently are C₁-C₆ alkyl, C₁-C₆ alkenyl, C₂-C₆ alkynyl, and R₁ and R₂ together with the carbon to which they are attached, and R₃ and R4 together with the carbon to which they are attached, can complete a carbocyclic ring having from 3 to 6 carbons;

Y1 and Y2 independently are COOH, CHO, tetrazole, and COOR₅ where R₅ is C₁-C₆ alkyl, C₂-C₆ alkenyl, C₂-C₆ alkynyl; and

where the alkyl, alkenyl, and allqynyl groups may be substituted with 1 or 2 groups selected from halo, hydroxy, C₁-C₆ alkoxy, and phenyl.

Preferred compounds to be employed in this invention have the above formula wherein n and m are the same integer, and wherein R₁, R₂, R₃, and R4 each are alkyl.

Further preferred are compounds wherein Y1 and Y2 independently are COOH or COOR₅ where R₅ is alkyl.

The most preferred compounds to be employed have the Formula II

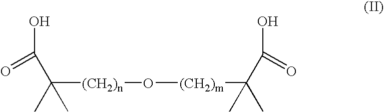

wherein n and m are each an integer selected from 2, 3, 4, or 5, ideally 4 or 5.

An especially preferred compound has the Formula III

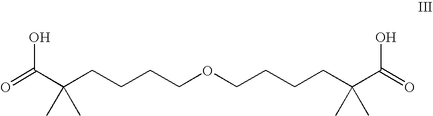

The combinations of this invention can also employ the pharmaceutically acceptable salts of the acids of the above formulas. The monocalcium salt of the compound of Formula III is now referred to as “CI-1027“ and is currently being developed for clinical treatment of dyslipidemias, and is especially preferred according to this invention.

According to this invention, the foregoing carboxyalkylethers are used in combination with an ACAT inhibitor to treat dyslipidemias and to improve ischemic syndrome.

Compounds which effectively inhibit the enzyme acyl-coenzyme A:cholesterol acyltransferase (ACAT) prevent the intestinal absorption of dietary cholesterol into the blood stream, or the reabsorption of cholesterol which has been previously released into the intestine through the body's own regulatory action. The ACAT inhibiting compounds provide treatment of hypercholesterolemia and atherosclerosis. Such compounds include, for example, a compound of Formula IV

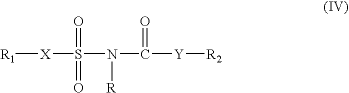

or a pharmaceutically acceptable salt thereof wherein:

X and Y are selected from oxygen, suffir, and (CR′R″)n, wherein n is an integer

of from 1 to 4 and R″ and R″ are each independently hydrogen, alkyl,

alkoxy, halogen, hydroxy, acyloxy, cycloalkyl, phenyl, optionally

substituted or R″ and R″ together form a spirocycloalkyl or a carbonyl;

with the proviso at least one of the X and Y is (CR′R″)n and with the

further proviso when X and Y are both (CR′R″)n, and R′ and R″

are hydrogen and n is 1, R₁ and R₂ are aryl;

R is hydrogen, a straight or branched alkyl of from 1 to 8 carbon atoms or benzyl;

R₁ and R₂ are each independently selected from

(a) phenyl or phenoxy each of which is unsubstituted or is substituted with 1 to 5 substituents selected from phenyl,

an alkyl group having from 1 to 6 carbon atoms and which is straight or branched,

an alkoxy group having from 1 to 6 carbon atoms and which is straight or branched;

phenoxy,

hydroxy,

fluorine,

chlorine,

bromine,

nitro,

trifluoromethyl,

-COOH,

-COOalkyl wherein alkyl has from 1 to 4 carbon atoms and is straight or branched,

-(CH₂)pNR₃R4 wherein p is 0 or 1, and each of R3 and R4 is selected from hydrogen or a straight or branched allyl group having 1 to 4 carbon atoms;

(b) 1- or 2-naphthyl unsubstituted or substituted with from 1 to 3 substituents selected from

phenyl,

an alkyl group having from 1 to 6 carbon atoms and which is straight or branched,

an alkoxy group having from 1 to 6 carbon atoms and which is straight or branched;

hydroxy,

phenoxy,

fluorine,

chlorine,

bromine,

nitro,

trifluoromethyl,

-COOH,

-COOalkyl wherein alkyl has from I to 4 carbon atoms and is straight or branched,

-(CH₂)pNR3R4 wherein p, R₃, and R4 have the meanings defined above;

(c) arylaklyl;

(d) a straight or branched alkyl chain having from 1 to 20 carbon atoms and which is saturated or contained from 1 to 3 double bonds; or

(e) adamantyl or a cycloalkyl group wherein the cycloalkyl moiety has from 3 to 6 carbon atoms.

The ACAT inhibitor for use in the novel method may be selected 1ID from any effective compound, especially compounds of Formula IV above, especially sulfamic acid, [[2,4,6-tris(methylethyl)-phenyljacetyl]-, 2,6-bis[(1-methylethyl)phenyl ester; 2,6-bis(l-methylethyl)phenyl- [[2,6-bis(1-methylethyl)-phenyl]sulfonyl]carbamate monosodium salt; N-(2,6-di-isopropyl-phenyl)-2-phenyl-malonamic acid dodecyl ester; 15 N-(2,6-diisopropyl-phenyl)-2-(2-dodecyl-2H-tetrazol-5-yl)-2-phenyl- acetamide; 2,2-dimethyl-N-[2,4,6-trimethoxyphenyl)-docecanamide; and N-[2,6-bis(l -methylethyl)phenyl]-N′-[[l -[4-(dimethyl-amino) phenyl]cyclopentyl]methyl urea monohydrochIoride.

One especially usefhll ACAT inhibitor that is disclosed in U.S. Pat. No. 5,491,172 is 2,6-bis(l-methylethyl) phenyl[[2,4,6-tris(l - methylethyl)phenyljacetyl]sulfamate. This compound is called avasimibe or CI-1011.

### DETAILED DESCRIPTION OF THE INVENTION

According to this invention, a carboxyalkylether is used in combination with an ACAT inhibitor to treat dyslipidemias and to improve ischemic syndromes in patients in need of treatment. The compounds can be employed individually or can be combined in a single formulation, for example as a tablet, capsule, syrup, solution, as well as controlled release formulations. In a preferred embodiment, the carboxyalkylether and the ACAT inhibitor are formulated individually and administered in the same manner that each is normally used clinically.

The dosage of each agent will vary depending upon the severity of the disease, the frequency of administration, the particular agents and combinations utilized, and other factors routinely considered by an attending medical practitioner. The carboxyalkylether normally will be administered at a daily dose of from about 0.25 mg to about 1500 mg, typically about 150mg to about 1000 mg. A typical dosage for CI-1027, for example, will be about 150 to about 900 mg per day. The ACAT inhibitor will normally be administered at doses from about 50 mg to about 1500 mg per day, and more typically from about 100 mg to about 600 mg per day. A preferred ACAT inhibitor is CI-1011, and it will be employed at doses from about 50 mg to about 750 mg per day.

The invention provides compositions of a carboxyalkylether and an ACAT inhibitor, and a method of treating dyslipidemia and controlling ischernic syndromes comprising administering to a patient in need of treatment an effective amount of a carboxyalkylether and an effective amount of an ACAT inhibitor. The amounts to be used are those that are effective for achieving an improvement in ischemic syndromes and/or dyslipidemias. When the carboxyalkylether and ACAT inhibitor are formulated together, the compositions will contain about one to about 1000 parts by weight of carboxyalkylether, and about 1000 to about one part by weight ACAT inhibitor. For example, a typical composition of CI-1027 and CI-I011 will contain about 500 mg of CI-1 027 and about 500 mg of CI-1011. Such combination will be administered to an adult patient about once each day to achieve a desired control of dyslipidemia and ischemic syndromes.

The various acute ischemic syndromes that may be treated by the method of the present invention include: angina pectoris, coronary artery disease (CAD), hypertension, cerebrovascular accidents, transient ischemic attacks, chronic obstructive pulmonary disease, chronic hypoxic lung disease, pulmonary hypertension, renal hypertension, chronic renal disease, microvascular complications of diabetes, and vaso-occlusive complications of sickle cell anemia.

For preparing the pharmaceutical compositions from the compounds of this invention, inert, pharmaceutically acceptable carriers can be either solid or liquid. Solid form preparations include powders, tablets, dispersible granules, capsules, and cachets.

A solid carrier can be one or more substances which may also act as diluents, flavoring agents, solubilizers, lubricants, suspending agents, binders, or tablet disintegrating agents; it can also be encapsulating material.

In powders, the carrier is a finely divided solid which is in a mixture with a finely divided active component. In tablets, the active component is mixed with the carrier having the necessary binding properties in suitable proportions and compacted in the shape and size desired. - Powders and tablets preferably contain between about 5% to about 70% by weight of the active ingredient. Suitable carriers are magnesium dicarbonate, magnesium stearate, talc, lactose, sugar, pectin, dextrin, starch, tragacanth, methyl cellulose, sodium carboxymethyl cellulose, a low-melting wax, cocoa butter, and the like.

The term “preparation” is intended to include the formulation of the active compound with encapsulating material as a carrier providing a capsule in which the active component (with or without other carriers) is surrounded by a carrier, which is thus in association with it. In a similar manner cachets or transdermal systems are also included.

Tablets, powders, cachets, and capsules can be used as solid dosage forms suitable for oral administration.

Liquid form preparations include solutions, suspensions, or emulsions suitable for oral administration. Aqueous solutions for oral administration can be prepared by dissolving the active compound in water and adding suitable flavorants, coloring agents, stabilizers, and thickening agents as desired. Aqueous suspensions for oral use can be made by dispersing the finely divided active component in water together with a viscous material such as natural or synthetic gums, resins, methyl cellulose, sodium carboxymethylcellulose, and other suspending agents known to the pharmaceutical formulation art. Preferably, the pharmaceutical preparation is in unit dosage form. In such form, the preparation is divided into unit doses containing appropriate quantities of the active component. The unit dosage form can be a packaged preparation containing discrete quantities of the preparation, for example, packeted tablets, capsules, and powders in vials or ampoules. The unit dosage form can also be a capsule, cachet, or tablet itself, or it can be the appropriate number of these packaged forrns.

The dosage forms are well within the skill of a physician who will be familiar with such factors as time of day and other pertinent considerations.

As noted above, any ACAT inhibitor can be used in combination with any carboxyalkylether. A preferred ACAT inhibitor is CI-1011.

Other ACAT inhibitors are 2,6-bis-(1-methylethyl)-phenyl[[2,6-bis(l-methylethyl) phenoxy]-sulfonyl]-carbamate monosodium salt; and similar compounds are disclosed in U.S. Pat. No. 5,245,068; N-(2,6-diisopropyl-phenyl)-2-phenyl-malonamic acid dodecyl ester; and similar compounds are disclosed in U.S. Pat. No. 5,420,339; N-(2,6-diisopropyl-phenyl)-2-(2-dodecyl-2H-tetrazol-5-yl)-2-phenyl-acetamide; and similar compounds are disclosed in U.S. Pat. No.5,366,987 and divisional 5,441,975; N-[2,6-bis (I-methylethyl)phenyl]-N′-[[l-[4-(dimethylamino)phenyl]cyclo-pentyl]methyl]urea monohydrochloride disclosed in U.S. Pat. No. 5,015,644; and 2,2-dimethyl-N-(2,4,6-trimethoxyphenyl) docecanamide and similar compounds disclosed in the U.S. Pat. No. 4,716,175.

The lipid modifying and antiatherosclerotic action of 2,6-bis(l-methylethyl) phenyl[[2,4,6-tris(l -methylethyl)phenyl]acetyl]sulfamate (Cl-1011), CI-1027, and the combination of both compounds was assessed in a rabbit cuff model of atherosclerosis. The assay was carried out according to the following protocol.

A chronic mechanical injury is created by placing a sterile, flexible polyethylene or silicone cuff around the left carotid artery of adult rabbits. The insult can be further enhanced by the inclusion of inflammatory agents, cytoldnes, or oxidative stress products dispersed in pluronic gel that would be encased by the collar. The periarterial sheath will be approximately 1 cm in length with a 1.8 mm inside diameter, and 3 mm outside diameter. The collars are biologically inert and will not obstruct blood flow.

There are two procedural paradigms using this cuff model. The progression study can include an acclimation period to the atherogenic diet for 1 to 2 weeks prior to surgical manipulation. The rabbits will be allocated according to their plasma total cholesterol levels. Drug treatment would commence at the time of surgery and would continue for 3 weeks. In the regression study, the surgery would be concurrent with cholesterol feeding. Lesions would develop for an additional 3 weeks. The animals will then be allocated into treatment groups based on their plasma total cholesterol levels. Afterwards, drug treatment would follow for the final 3 weeks. All compounds to be evaluated will be administered as an admixture to the cholesterol diet.

Results are summarized in Table 1 below.

TABLE 1
Effects of Treatment on Lipid and Lipoprotein Parameters in the
Chow-Fed Rat

| Treatment | TG | Cholesterol | VLDL | LDL | HDL | HDL/VLDL + LDL |
|---|---|---|---|---|---|---|
| Control | 126 ± 9.6 | 49 ± 2.3 | 5 ± 0.2 | 9.4 ± 0.5 | 33 ± 2.4 | 2.3 ± 0.2 |
| CI-1027 (mg/kg) | | | | | | |
| 3 | 70 ± 2.8 | 40 ± 1.4 | 2.7 ± 0.3 | 9.0 ± 0.5 | 28 ± 1.0 | 2.4 ± 0.1 |
| 10 | 60 ± 8.8 | 89 ± 8.5 | 2.8 ± 0.8 | 9.1 ± 1.1 | 76 ± 7.1 | 6.8 ± 0.7 |
| 30 | 27 ± 2.0 | 119 ± 21 | 0.7 ± 0.2 | 17.6 ± 6.1 | 100 ± 14.8 | 9.4 ± 2.7 |
| Avisimbe (mg/kg) | | | | | | |
| 10 | 85 ± 16.8 | 36 ± 2.3 | 3.9 ± 0.7 | 12.0 ± 1.1 | 19.5 ± 1.7 | 1.2 ± 0.1 |
| 30 | 65 ± 12.9 | 37 ± 2.9 | 2.1 ± 0.6 | 12.0 ± 0.8 | 22.2 ± 1.8 | 1.6 ± 0.1 |
| Avisimbe + CI-1027 | | | | | | |
| 3, 10 | 42 ± 7.0 | 42 ± 6.1 | 1.7 ± 0.4 | 8.7 ± 1.3 | 31 ± 4.7 | 3.1 ± 0.3 |
| 10, 10 | 26 ± 3.9 | 40 ± 6.3 | 0.6 ± 0.1 | 2.5 ± 0.6 | 36 ± 5.8 | 13.3 ± 2.1 |
| 30, 10 | 20 ± 1.4 | 77 ± 11.2 | 0.12 ± 0.1 | 4.3 ± 0.9 | 72 ± 10.5 | 18.3 ± 3.1 |

“TG” = Triglyceride; “Cholesterol” = Total cholesterol; “VLDL” = Very low-density cholesterol; “LDL” = Low-density cholesterol; “HDL” = High-density cholesterol; “HDL/VLDL + LDL” = HDL divided by the sum of VLDL and LDL.

The method of treating dyslipidemia and ischemic syndromes employing a combination of a carboxyalkylether and an ACAT inhibitor also will be established in a long-term controlled clinical evaluation. The study will determine the efficacy and safety of the ACAT inhibitor- alone and in combination with the carboxyalkylether for the treatment of dyslipidemias and ischernic syndromes.

This study will target the segment of the population at risk for developing a heart attack.

What is claimed is:

1. A pharmaceutical composition comprising:
a. an amount of carboxyalkylether or pharmaceutically acceptable acid addition salt thereof;
b. an amount of an ACAT inhibitor or a pharmaceutically acceptable salt thereof; and
c. a pharmaceutically acceptable carrier or diluent.

2. A pharmaceutical composition of claim 1 wherein said ACAT inhibitor is a compound having Formula IV 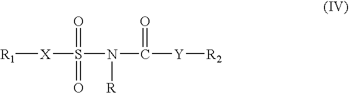
or a pharmaceutically acceptable salt thereof wherein:
X and Y are selected from oxygen, sulfur, and (CR′R″)ₙ, wherein n is an integer of from 1 to 4 and R′ and R″ are each independently hydrogen, alkyl, alkoxy, halogen, hydroxy, acyloxy, cycloalkyl, phenyl, optionally substituted or R′ and R″ together form a spirocycloallkyl or a carbonyl;
with the proviso at least one of the X and Y is (CR′R″)n and with the further proviso when X and Y are both (CR′R″)n, and R′ and R″ are hydrogen and n is 1, R₁ and R₂ are aryl;
R is hydrogen, a straight or branched alkyl of from 1 to 8 carbon atoms or benzyl;
R₁ and R₂ are each independently selected from
(a) phenyl or phenoxy each of which is unsubstituted or is substituted with 1 to 5 substituents selected from phenyl,
an alkyl group having from 1 to 6 carbon atoms and which is straight or branched,
an alkoxy group having from 1 to 6 carbon atoms and which is straight or branched;
phenoxy,
hydroxy,
fluorine,
chlorine,
bromine,
nitro,
trifluoromethyl,
-COOH,
-COOalkyl wherein alkyl has from 1 to 4 carbon atoms and is straight or branched,
-(CH₂)pNR₃R4 wherein p is 0 or 1, and each of R₃ and R4 is selected from hydrogen or a straight or branched alkyl group having I to 4 carbon atoms;
(b) 1- or 2-naphthyl unsubstituted or substituted with from 1 to 3 substituents selected from phenyl,
an alkyl group having from 1 to 6 carbon atoms and which is straight or branched,
an alkoxy group having from I to 6 carbon atoms and which is straight or branched;
hydroxy,
phenoxy,
fluorine,
chlorine,
bromine,
nitro,
trifluoromethyl,
-COOH,
-COOalkyl wherein alkyl has from 1 to 4 carbon atoms and is straight or branched,
-(CH₂)ₚNR₃R₄ wherein p, R₃, and R4 have the-meanings defined above;
(c) arylaklyl;
(d) a straight or branched alkyl chain having from 1 to 20 carbon atoms and which is saturated or contained from 1 to 3 double bonds; or
(e) adamantyl or a cycloalkyl group wherein the cycloalkyl moiety has from 3 to 6 carbon atoms.

3. A pharmaceutical composition of claim 2 wherein said ACAT inhibitor in [(2,4,6-triisopropyl-phenyl)-acetyl]-sulfamic acid 2,6-diisopropyl-phenyl ester.

4. A pharmaceutical composition of claim 3 comprising a carboxyalkylether compound of Formula I: 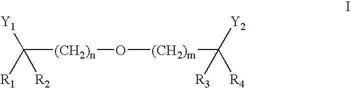
and the pharmaceutically acceptable salts thereof, wherein:
n and m independently are integers from 2 to 9;
R₁, R₂, R₃, and R4 independently are C₁ -C₆ alkyl, Cj -C₆ alkenyl, C₂-C₆ alkynyl, and R₁ and R₂ together with the carbon to which they are attached, and R₃ and R4 together with the carbon to which they are attached, can complete a carbocyclic ring having from 3 to 6 carbons;
Y₂ and Y₂ independently are COOH, CHO, tetrazole, and COOR₅ where R₅ is C₁-C₆ alkyl, C₂-C₆ alkenyl, C₂-C₆ alkynyl; and
where the alkyl, alkenyl, and alkynyl groups may be substituted with 1 or 2 groups selected from halo, hydroxy, C₁-C₆ alkoxy, and phenyl.

5. A pharmaceutical composition of claim 4 comprising [(2,4,6-triisopropyl-phenyl)-acetyl3-sulfamic acid 2,6-diisopropyl-phenyl ester and 6,6′-oxybis(2,2-dimethylhexanoic acid) or pharmaceutically.acceptable salt thereof.

6. A first pharmaceutical composition for use with a second pharmaceutical composition for achieving an improved dyslipidemia effect and/or improved ischemic syndrome effect in a mammal suffering from dyslipidemia or ischemic syndrome or suspected of developing dyslipidemia or ischemic syndrome, which effects are greater than the sum of the effects achieved by administering said first and second pharmaceutical compositions separately, and which second pharmaceutical composition comprises an amount of a carboxyaulcylether or a pharmaceutically acceptable acid addition salt thereof and a pharmaceutically acceptable carrier or diluent, said first pharmaceutical composition comprising an amount of an ACAT inhibitor or a pharmaceutically acceptable salt thereof and a pharmaceutically acceptable carrier or diluent.

7. A composition of claim 6 wherein said ACAT inhibitor is [(2,4,6-triisopropyl-phenyl)-acetyl]-sulfamic acid 2,6-diisopropyl-phenyl ester.

8. A composition of claim 7 wherein said second pharmaceutical composition comprises 6,6′-oxybis(2,2-dimethylhexanoic acid) calcium salt.

9. A first pharmaceutical composition for use with a second pharmaceutical composition for achieving an improved ischemic syndrome in a mammal suffering from ischemic syndrome or suspected of developing an ischemic syndrome, which effects are greater than the sum of the improvements in ischemic syndromes achieved by administering said first and second pharmaceutical compositions separately and which second pharmaceutical composition comprises an amount of an ACAT inhibitor or a pharmaceutically acceptable salt thereof and a pharmaceutically acceptable carrier or diluent, said first pharmaceutical composition comprising an amount of a compound of Formula II 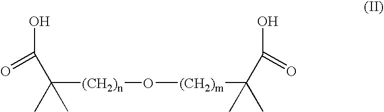
or a pharmaceutically acceptable acid addition salt thereof and a pharmaceutically acceptable carrier or diluent.

10. A composition of claim 9 wherein said ACAT inhibitor is [(2,4,6- triisopropyl-phenyl)-acetyl]-sulfamic acid 2,6-diisopropyl-phenyl ester.

11. A composition of claim 10 comprising 6,6′-oxybis(2,2-dimethylhexanoic acid) or salt thereof.

12. A first pharmaceutical composition for use with a second pharmaceutical composition for managing ischernic syndromes in a mammal, which effect is greater than the sum of the improved ischemic syndrome control achieved by administering said first and second pharmaceutical compositions separately, and which second pharmaceuacal composition comprises an amount of an ACAT inhibitor or a pharmaceutically acceptable salt thereof and a pharmaceutically acceptable carrier or diluent, said first pharmaceutical composition comprising an amount of a carboxyalkylether or a pharmaceutically acceptable acid addition salt thereof and a pharmaceutically acceptable carrier or diluent.

13. A composition of claim 12 wherein said ACAT inhibitor is [(2,4,6-triisopropyl-phenyl)-acetyl]-sulfamic acid 2,6-diisopropyl-phenyl. ester.

14. A composition of claim 13 comprising 6,6′-oxybis(2,2-dimethylhexanoic acid) or salt thereof.

15. A kit for achieving a therapeutic effect in a mammal comprising:
an amount of a carboxyalkylether or a pharmaceutically acceptable acid addition salt thereof and a pharmaceutically acceptable carrier or diluent in a first unit dosage form;
b. an amount of an ACAT inhibitor or a pharmaceutically acceptable salt thereof and a pharmaceutically acceptable carrier or diluent in a second unit dosage form; and
c. container means for containing said first and second dosage forms.

16. A kit of claim 15 wherein said ACAT inhibitor is [(2,4,6-triisopropyl-phenyl)-acetyl]-sulfamic acid 2,6-diisopropyl-phenyl ester.

17. A kit of claim 16 comprising a carboxyalkylether of Formula I.

18. A kit of claim 17 employing 6,6′-oxybis(2,2-dimethylhexanoic acid) or salt thereof.

19. A kit of claim 15 wherein said therapeutic effect is treatment of ischemic syndromes.

20. A composition comprising an effective amount of 6,6′-oxybis(2,2-dimethylhexanoic acid) or a pharmaceutically acceptable salt thereof and an effective amount of [(2,4,6-triisopropyl-phenyl)-acetyl]-sulfamic acid 2,6-diisopropyl-phenyl ester.
